(19) United States
(12) Patent Application Publication
(10) Pub. No.: US 2011/0263044 A1
(43) Pub. Date: Oct. 27, 2011

(54) DEVICE AND METHOD FOR THE AUTOMATIC DETECTION OF BIOLOGICAL PARTICLES

(75) Inventors: Ulrich Reidt, Schwalmstadt (DE); Alois Friedberger, Oberpframmern (DE); Christoph Heller, Taufkirchen (DE); Thomas Ziemann, Inning am Holz (DE); Harald Waltenberger, Ismaning (DE); Günter Müller, Bernried (DE)

(73) Assignee: EADS DEUTSCHLAND GMBH, Ottobrunn (DE)

(21) Appl. No.: 13/056,355

(22) PCT Filed: Jul. 22, 2008
(86) PCT No.: PCT/EP09/59446

§ 371 Date: May 23, 2011

(30) Foreign Application Priority Data
Jul. 31, 2008 (DE) 10 2008 035 771.5

(51) Int. Cl. G01N 33/53

(52) U.S. Cl. 436/501; 422/69

(57) ABSTRACT

An apparatus and a method for automatic detection of particles, in particular, biological particles such as micro-organisms. The apparatus has a device for binding the particles to separating particles which can be bound selectively to the particles. The apparatus further includes a separating device for extraction of the separating particles with bound particles from a collecting fluid, and a detection unit for detecting a number and/or concentration of the particles separated in this manner. The device for binding the particles to the separating particles includes a collecting device for collecting a collecting fluid including the separating particles and the particles that are provided by a particle-fluid mixture.

The invention relates to an apparatus and a method for automatic detection of biological particles.

U.S. Pat. No. 6 268 143 B1 and U.S. Pat. No. 5,972,721 describe methods and apparatuses for automatic extraction and detection of biological particles, in particular of micro-organisms such as bacteria. In this case, the principle of paramagnetic separation or of immuno-magnetic investigation is used. Separating particle bodies are used for this purpose, which are coated such that they bind very specific particles to be investigated to them, composed of paramagnetic material. These separating particle bodies—called beads—can be fixed in a magnetic field and can thus be extracted. The specific particles which adhere thereto are therefore also extracted.

Further examples and specific beads are described in WO 03/010563 A2, WO 2006/112771 A1, WO 2006/021410 A1, EP 0 687 501 A2, U.S.Pat. No. 6,207,463 B1 and U.S. Pat. No. 5,821,066 A.

The paramagnetic separation is also explained in more detail in the following publications:
Olsvik O, Popovic T, Skjerve E, Cudjoe K S, Homes E, Ugelstad J, Uhlen M Magnetic separation techniques in diagnostic microbiology.
Clin. Microbiol. Rev. 1994 January; 7(1):43-54. Review and
Mullane N R, Murray J, Drudy D, Prentice N, Whyte P, Wall P G, Parton A, Fanning S.
Detection of Enterobacter sakazakii in dried infant milk formula by cationic-magnetic-bead capture.
Appl. Environ. Microbiol. 2006 September; 72(9):6325-30.

At present, however, no capability exists for fully automatic and rapid detection of micro-organisms and biological particles from gases or air.

At the moment, micro-organisms are detected from the air or from a gas by means of so-called air samplers, which are fitted with nutrient media. After the samples have been taken, these must be incubated for several hours or days before detection is possible. Systems for checking and enrichment of micro-organisms from the air into a liquid are likewise known, but these systems lack the capability for fully automatic extraction and detection.

Examples of the abovementioned air samplers can be found in U.S. Pat. No. 5,902,385 and U.S. Pat. No. 5,904,752.

The principle of air sampling is explained in more detail in the following publications:
Hogan C J JR, Kettleson E M, Lee M H, Ramaswami B, Angenent L T, Biswas P.,
Sampling methodologies and dosage assessment techniques for submicrometre and ultrafine virus aerosol particles.
J. Appl. Microbiol. 2005; 99(6):1422-34.
Agranovski I E, Agranovski V, Grinshpun S A, Reponen T, Willeke K,
Collection of airborne microorganisms into liquid by bubbling through porous medium.
Aerosol Science and Technology, Volume 36, Number 4, 1 April 2002, pp. 502-509(8) and
Lödding H, Koch, W, Möhlmann C, Kolk A,
Sammelverhalten von Impingern als Bioaerosolsammler
Gefahrstoffe-Reinhaltung der Luft-Ausgabe 7-8/2007 [Collection behavior of impingers as bio-aerosol collectors, hazardous substance purification of the air—ssues July-August 2007 ]

The object of the invention is to provide an apparatus and a method for detection of particles in a particle/fluid mixture, which apparatus can be operated and which method can be carried out fully automatically, can be used universally, and can be implemented in a compact, preferably mobile, system of simple design.

This object is achieved by an apparatus having the features of patent claim 1, and by a method having the steps of the other independent claim.

Advantageous refinements of the invention are the subject matter of the dependent claims.

A particularly preferred system, as proposed here, allows rapid and fully automatic enrichment, extraction and detection of micro-organisms (for example bacteria, protozoa, moulds, viruses) and biological particles (for example spores). The enrichment, extraction and detection can be carried out both from gases, in particular from the air, and from liquids. In addition to detection of biological materials, enrichment, extraction and detection of non-biological and/or synthetic materials are also possible, in particular explosives, liquid explosives and drugs.

The novel technique described here proposes, in particular, the use of paramagnetic small spheres (so-called beads) in conjunction with a collecting device, in particular an air sampler. The beads are coated with antibodies, which in turn can bind molecules or particles of biological or non-biological origin. The extreme concentration and immobilization of the beads which have been loaded in this way is achieved by the development of specific enrichment techniques. Furthermore, it is proposed that the concentration process be followed by fully automatic extraction and detection of the bound molecules or particles. The high concentration level allows highly sensitive detection of the analytes.

In particular, the following advantages are achieved by the invention or its advantageous refinements:
rapid and sensitive detection of micro-organisms and other hazardous substances (in particular biological toxins) and of explosives from a gaseous phase, in particular air;
rapid detection of micro-organisms and other hazardous substances from liquids and liquid foodstuffs of all types;
combination and automation of the three areas of enrichment, extraction and detection in a standard, compact and mobile system, and/or
rapid detection of pathogens from body fluids, in particular blood, saliva, lacrimal fluid and urine (medical diagnosis).

Exemplary embodiments of the invention will be explained in more detail in the following text with reference to the attached drawing, in which:

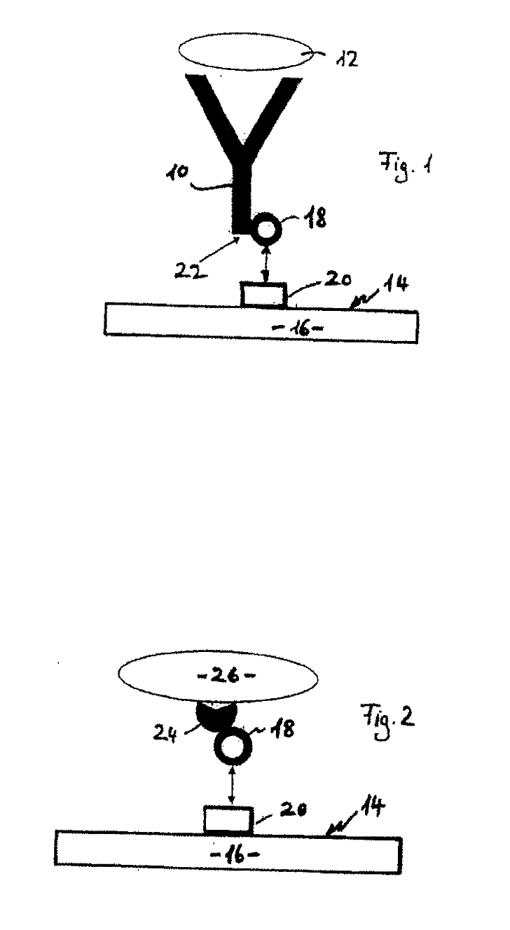

FIG. 1 shows a schematic illustration in order to explain the identification and binding of particles (antigens) by means of antibodies which have been immobilized on a bead surface by means of biotin and streptavidin;

FIG. 2 shows a schematic illustration in order to explain the identification and binding of bacteria by means of phage proteins which have been immobilized on a bead surface via biotin and streptavidin;

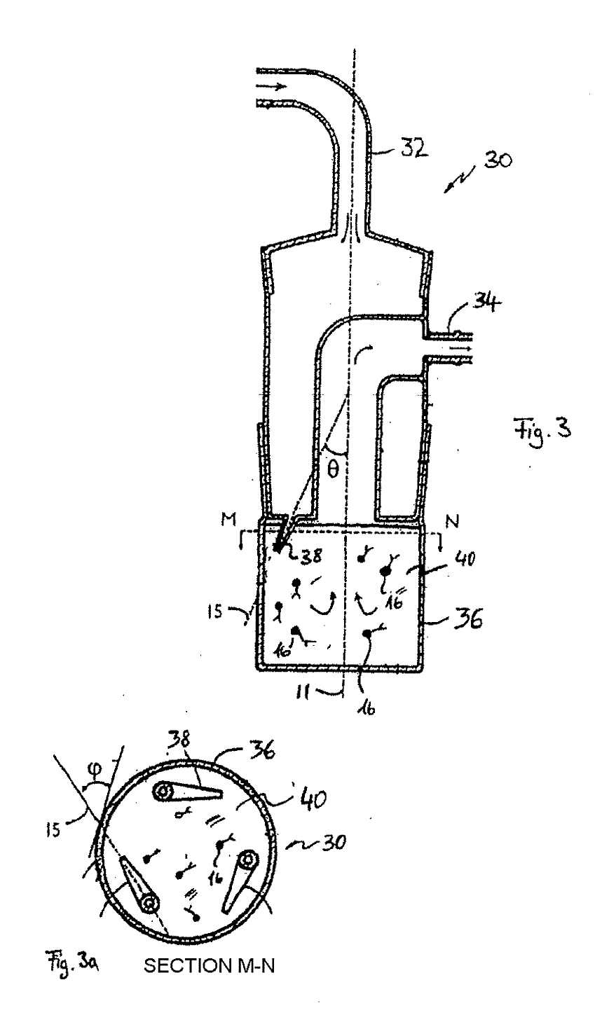

FIGS. 3, 3a show various section views of an air sampler, which is known per se and can be used in the system proposed here, in an appropriate application;

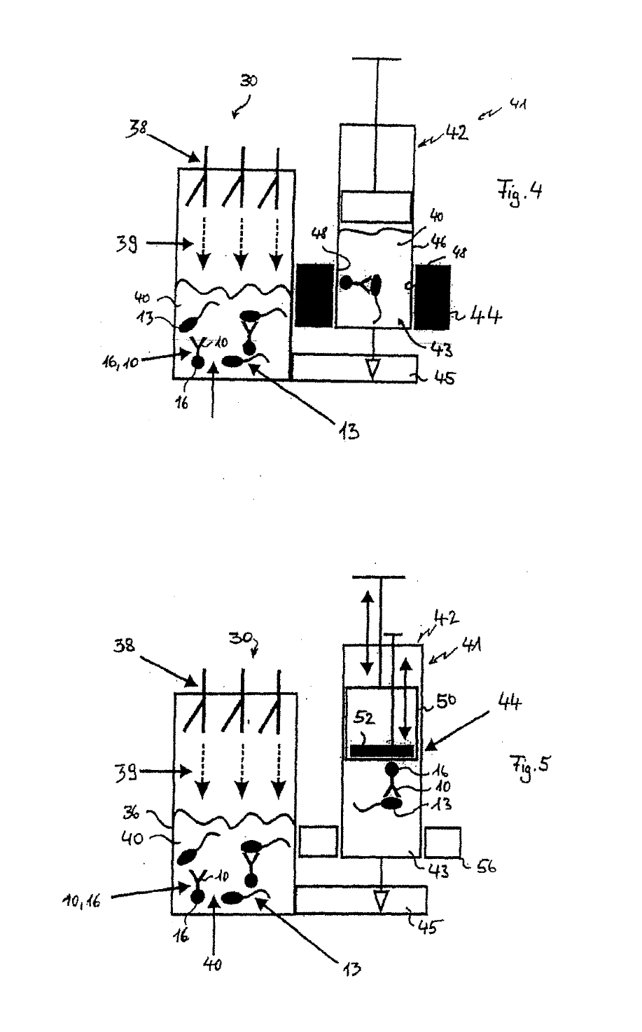

FIG. 4 shows a schematic illustration of enrichment of the paramagnetic beads by means of an external magnet;

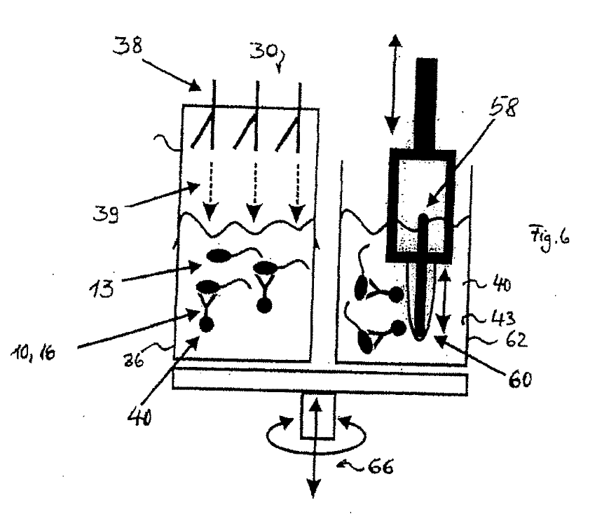

FIG. 5 shows a schematic illustration of enrichment of the paramagnetic beads by means of a magnetic piston;

FIG. 6 shows a schematic illustration of enrichment of the paramagnetic beads via an expandable membrane;

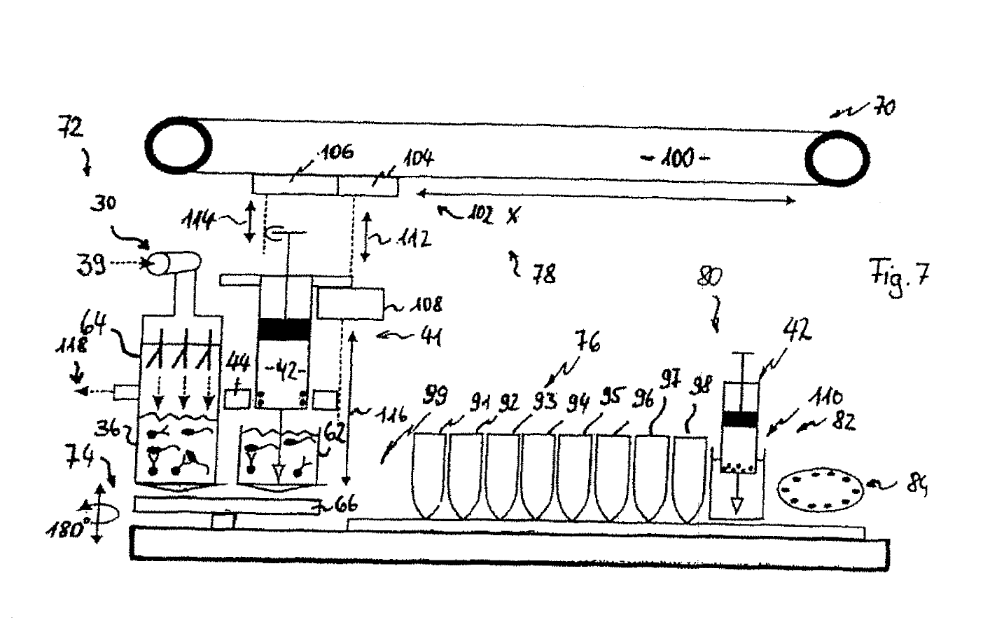

FIG. 7 shows a side view of an overall system as an apparatus for detection of particles;

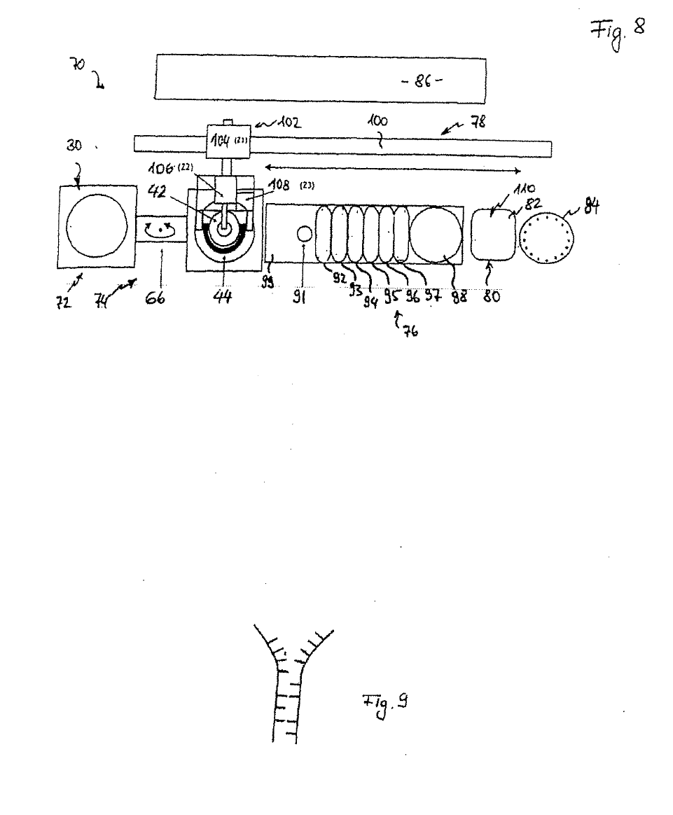

FIG. 8 shows a plan view of the overall system;

FIG. 9 shows a DNA sequence based on the model of a zip fastener,

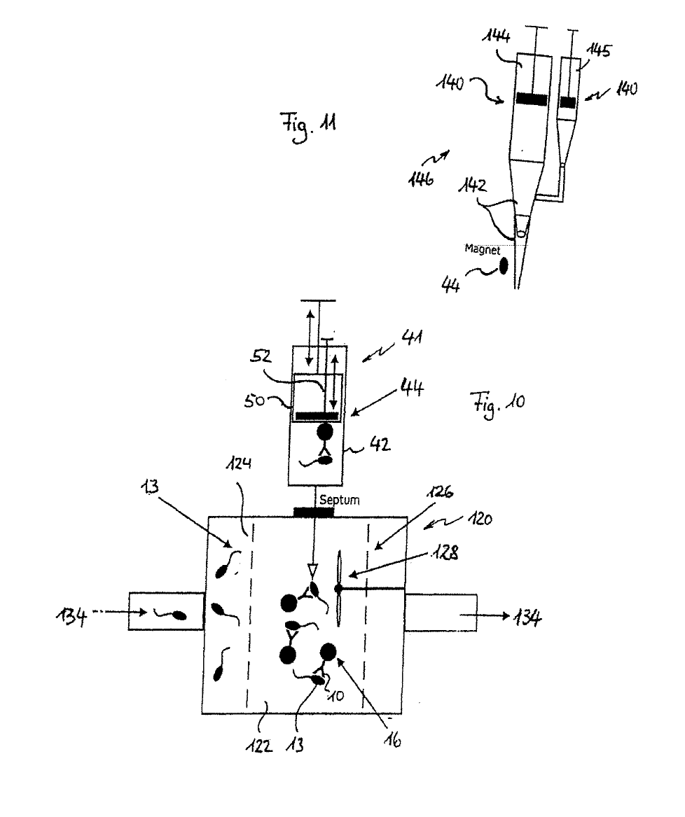

FIG. 10 shows a schematic illustration of the enrichment of particles from liquids via a flow cell; and

FIG. 11 shows a schematic illustration of two microliter pipettes linked to one another.

The following text describes in more detail exemplary embodiments of a system which is intended to be used for fully automatic sampling, enrichment, extraction and analysis of gases and liquids.

### I. Introduction:

The primary aim is to detect all particles, in particular bacteria, viruses, spores, protozoa and biological toxins. The appliance described here can equally well be used for analysis of biological and non-biological substances. The only precondition is the presence of specific binding molecules (for example antibodies) with adequate affinity. Since an antibody can be formed against virtually any substance, the appliance described here can be used to detect many substances.

II. Paramagnetic Beads with Immobilized Antibodies or Other Binding Proteins:

In one preferred refinement, paramagnetic small spheres (beads) are used as separating particle bodies for separation of the particles of interest here in the novel technology, which beads are fitted with antibodies and to which the particles or micro-organisms bind with high affinity. In general, the beads themselves have a size in the range from below 0.5 μm to 10 μm, and consist predominantly of a paramagnetic core and a shell composed of silica, latex or polystyrene.

Various methods can be used to fit the beads with antibodies (so-called coating):

a) passive adsorption (for example via hydrophobic interactions),

b) direct chemical coupling (crosslinking) via peptide binding or the like,

c) coupling via immobilized antibodies proteins, for example protein A and/or protein G and/or

d) coupling via biotin-streptavidin binding.

The technique mentioned in d) is illustrated in FIG. 1 and uses the high affinity (binding force) of two biological molecules to one another: biotin 18 and streptavidin 20. For this purpose, the antibody 10 is labeled with the molecule biotin 18 on the side averted from its specific binding site (the so-called F_{C} domain 22). At the same time, the protein streptavidin 20 is coupled to the surface 14 of the paramagnetic beads 16. This results in antibodies 10 being bound virtually irreversibly to the sphere surface 14.

After the antibodies 10 have been immobilized onto the paramagnetic beads 16, they can be used to “trap” the micro-organisms or particles. In this case, the antibodies bind to their so-called antigens 12 (the particles 13 to be detected); in particular, these are specific surface structures of micro-organisms.

As illustrated in FIG. 2, as an alternative to antibodies 10, certain phage proteins 24 can be used for specific identification and binding of some types of bacteria—a bacterium 26 is described as an example. Phages are viruses which exclusively infect bacteria and, with their specific envelope proteins, can dock on the bacterial surface. Biotechnological methods can be used to produce large quantities of these phage proteins 24, and likewise to label them with biotin 18. Analogously to the antibodies 10, these phage proteins 24 can likewise be coupled to streptavidin-coated paramagnetic beds 16, with the phage proteins 24 which have been bound in this way themselves interacting with the bacterial docking points (surface proteins on the bacteria 26).

### III. Sampling and Enrichment:

In normal environmental air, germs etc. are normally present only in very low concentrations. Enrichment is therefore necessary in order to detect airborne germs (or other particles and molecules). In the technology described here, this enrichment is carried out in three steps:
1) transferring the particles 13 from air into liquid,
2) binding of the particles 13 to paramagnetic beads 16, and
3) concentration of the beads 16 by means of a magnetic field.

In this case, steps 1) and 2) are carried out simultaneously.

The environmental air—air flow 38—is sucked in through the inlet 32 in a so-called air sampler 30 as illustrated in FIG. 3—as described and illustrated, for example, in U.S. Pat. No. 5,902,385, to which reference is expressly made with regard to further details—and the particles 13 located therein—in particular airborne germs—are transferred by means of special, high-efficiency, nozzles (approximately 90% yield) into a small liquid volume. By way of example, the air flow 39 through the sampler is 12.5 l/min; with a sampling time of, for example, 10 minutes, this therefore corresponds to a total volume of 125 l of air. If, for example, the volume of the collecting liquid is typically 5 ml, this results in a concentration by a factor of 25.000.

FIG. 3 and FIG. 3a show cross-sectional drawings, which illustrate the air sampler 30 with the inlet 32, the outlet 34, the collecting container 36 and tangential nozzles 38. FIG. 3a shows a cross section along the line M-N of FIG. 3. FIG. 3 also shows the centre axis 11 and a tangent 15, in order to illustrate the arrangement and alignment of the tangential nozzles 38.

The collecting liquid 40 in the collecting container 36 (collection vessel) has paramagnetic beads 16 as described above added to it in the system described here. The particles 30 (analytes), which are transferred from the air flow 39 (FIG. 4) into the collecting liquid 40, can bind to the specifically activated paramagnetic beads 16 during the sampling process. After the end of the sampling process, the beads 16 are attracted by means of a magnetic field, and are enriched in a small volume within the metering volume 43 of a metering unit 41 (FIG. 4). This volume is typically about 50 μL, thus achieving a further concentration by a factor of 100. Therefore, overall, the overall system allows, for example, airborne germs (or other particles) to be enriched by 2.5 million times.

The third step, that is to say the enrichment of the paramagnetic beads 16 from the collecting container 36 of the air sampler 30, can be carried out using three different technical processes, which will be explained in more detail in the following text.

### IIIA. Enrichment Via an External Magnet:

The following text refers to FIG. 4. A magnet 44 is fitted to the outer wall 46 on the metering unit 41—preferably a syringe 42.

The collecting liquid 40 with the paramagnetic beads 16 is drawn via an outlet channel 45 out of the air sampler 30 into the metering unit 41.

While the liquid 40 is flowing into the metering unit 41, the beads automatically accumulate on the inner wall 48 of the metering unit 41 in the area of the magnetic field.

The collecting liquid 40 is then forced out of the syringe 42 again. The beads 16 are held and held back by the magnet 44. The beads 16 can be washed by repeatedly receiving and discharging fresh buffer.

In the final wash step, as small a buffer volume as possible is left for the elution in the syringe 42, and the magnet 44 is moved away from the outer wall 46 of the metering unit 41.

After resuspension of the beads 16, they can now be transferred into another reaction vessel—not illustrated—for further processing. In order to ensure that the syringe 42 is emptied completely, a small volume of liquid can be received and eluted again.

### III.B. Enrichment Via a Telescopic Piston:

In the second process, which is illustrated in FIG. 5, the collecting liquid 40 is likewise drawn out of the air sampler 30 via an outlet channel 45 into the metering unit 41, preferably a syringe 42. The syringe 42 has hollow syringe piston 50, in which a second, magnetic piston—magnet piston 52—or plunger—can be moved up and down with the magnet 44 (telescopic principle).

While the collecting liquid 40 is being drawn, the magnet piston 52 is moved entirely into the syringe piston 50. The paramagnetic beads 16 can thus be enriched on the piston base 54.

The collecting liquid 40 can be replaced by a different liquid by repeatedly raising and lowering the syringe piston 50 (so-called washing).

Finally, a minimal liquid volume is drawn (for example 50 μl). For elution, the magnet piston 52 is drawn upward, as a result of which the magnetic field in the syringe 42 effectively disappears, and the beads 16 are detached from the piston base 54. This concept offers the advantage that the syringe 42 acts as a normal metering unit 41 when the magnet 44 is raised.

Furthermore, in the example described here, a decomposition module, in particular an ultrasound appliance 56, is fitted in the lower area of the metering unit 41. This is particularly preferably done in conjunction with the magnet piston 52, since there is then no need to fit a magnet in the lower area, creating space for the decomposition module.

### III.C. Enrichment Via a Magnet Which is Surrounded by an Expandable Membrane:

In a third process, which is illustrated in FIG. 6, for enrichment of the beads 16, the magnet 44 (preferably a bar magnet 58) is immersed directly in the collecting liquid 40. The magnet 44 is mounted such that it can move, and is protected by an expandable membrane 60. Once the beads 10 have attached themselves to the membrane 60, the magnet 44 is immersed in a new vessel 62 with a small liquid volume. The magnet 44 is moved away, and the beads 16 can detach themselves from the membrane 60. The use of ultrasound, which is transmitted to the bar magnet, makes it easier to detach the beads. The beads can also be detached more easily by using an external ultrasound appliance.

The air sampler 30 illustrated in FIGS. 3, 3a has two components, a collecting vessel—in the form of a collecting container 36—and an attachment with nozzles—nozzle attachment 64. The collecting container 36 can automatically be disconnected from the nozzle attachment 64, and pivoting, by use of a linear-movement/pivoting unit 66. This allows easy access to the collecting liquid 40.

FIG. 6 shows the process described in III.C. in combination with the linear-movement/pivoting unit 66. However, the linear-movement/pivoting unit 66 can likewise be used in conjunction with the processes described in III.A. and III.B. In consequence, there is no longer any need for a special outlet channel 45 from the collecting container 36.

After enrichment by means of one of the processes described above, the bead-bound particles 30 are now available for further analysis. By way of example, they are further broken down for molecular-biological detection, in particular PCR or hybridization.

### III.D Enrichment and Dispensation Via Two Microliters Pipettes Which are Connected to One Another.

When liquid is removed from the collecting vessel, relatively large volumes have to be handled (for example 5 ml), while the aim is to handle as small a volume as possible (for example 20 μl) after concentration. This is a very wide bandwidth (factor 250) between the volumes. It is very difficult to ensure high accuracy for both ranges. This can be achieved by the solution explained in the following text, one exemplary embodiment of which is illustrated in FIG. 11.

FIG. 11 shows an alternative embodiment of the metering unit 41. The use of a microliter pipette 140 with a lengthened pipette tip 142 and two suction units 144, 145 for different volume ranges allows not only the washing of the beads 16, but also accurate pipetting in small volumes. In this case as well, the beads 16 are retained on the inside of the pipette tip during rinsing, by means of a magnet 44 which can be pivoted in on the outside. The pipetting system 146 described here can be integrated in the overall system without any problems. Operation with the linear-movement/pivoting unit 66 is also possible.

### IV. Description of the Overall System:

The enrichment concepts described above can be linked to further elements to form an overall system. The overall system, which is illustrated in more detail in FIGS. 7 and 8, forms an apparatus 70 for automatic detection of, in particular, biological particles, and, as components, has a collecting device 72, a transfer unit 74, a metering unit 41, the magnet 44, a group 76 of reservoirs, a drive unit 78, a decomposition device 80, possibly with a temperature-control unit 82, a detection unit 84 and a control unit 86.

These possible components will be explained in more detail in the following text.

### IV.A. Collecting Device:

The air sampler 30, in particular an air sampler 30 from the SKC Company (see FIG. 3 and U.S. Pat. No. 5,902,385 and U.S. Pat. No. 5,904,752) or from the Bertin Company is preferably used as the collecting device 72. The air sampler 30 transfers particles 13, in particular micro-organisms (bacteria, viruses) and toxins from the gas phase into the collecting liquid 40.

### IV.B. Transfer Unit:

The transfer unit 74 preferably has the linear-movement/pivoting unit 66. Since the preferred air sampler 30 is of modular design, and in particular consists of at least two components, the nozzle attachment 64 can be disconnected, and the collecting container 36 can be transferred to the enrichment position. Here, the paramagnetic beads 16 can be absorbed and enriched using a process as described in section 3.

### IV.C. Metering Unit:

The metering unit 41 is preferably in the form of a syringe 42. The metering unit 41 is used to draw up the collecting liquid 40. The designs described in III.C. or IIID can also be used as a metering unit 41.

### IV.D. Separating Device—Magnet

The magnet 44 is used as a separating device, in order to concentrate the paramagnetic beads 16 in or on the metering unit 41. The beads 16 can thus be separated from the liquid surrounding them.

### IV.E. Group of Reservoirs:

The group 76 has a plurality of reservoirs (vessels) 91-98 with different liquids, which are required for processing the particles 13. In addition, a rest position 99 is provided. In particular, the following liquid reservoirs are provided:
solution with paramagnetic beads (first reservoir 91)
equilibration solution (second reservoir 92)
first decomposition solution (third reservoir 93)
second decomposition solution (fourth reservoir 94)
collecting liquid, for example water (fifth reservoir 95)
cleaning solution (sixth reservoir 96)
preservation solution (seventh reservoir 97)
waste vessel (eighth reservoir 98).

The reservoirs 91-98 are preferably aligned on one line—together with the rest position 99 and the collecting device 72. This allows the metering unit 41 to be moved between the reservoirs 91-98, and possibly the rest position 99 and the collecting device 72, linearly by means of a linear drive 100 of simple design.

Furthermore, the overall system can easily be extended or reduced in size (depending on the purpose).

### IV.F. to IV.I. Drive Unit:

The drive unit 78 has the following drives, which will be explained in F) to I) in the following text:

F) a linear drive 100 with a unit 102 for holding the metering unit 41 for control of all positions (preferably in only one dimension, in this case in the X direction);

G) a first movement unit (first motor 104) for movement of the metering unit (preferably for movement of the syringe 42) in the Z direction—first movement 112—;

H) a second movement unit (second motor 106) for liquid metering (preferably for movement of a syringe piston 50)—second movement 114—and

I) a third movement unit (third motor 108) for moving the magnet 44 towards or away (for example in the Z direction)—third movement 116—.

### IV.J. Decomposition Device:

The decomposition device 80 preferably has the ultrasound appliance 56, as mentioned above, in particular in the form of an ultrasound bath 110, for the mechanical decomposition of the particles, in particular micro-organisms. The ultrasound bath 110 is filled with liquid, and the metering unit 41 can be immersed in this liquid. In a second function, the ultrasound bath 110 can be used, with a low power level, for resuspension of the paramagnetic beads 16.

IV.K. Decomposition Device with Temperature-Control Unit and Temperature Control of the Reagents:

In the illustrated example, the decomposition device 86 also has a first temperature-control unit 82, which can be operated together with or separately from the ultrasound bath 110. The heat-treatment unit 82 is used to assist biochemical methods for decomposition of the particles 13, in particular micro-organism (for example enzymatic digestion). Thermal decomposition processes are also possible, close to the boiling point, by means of the temperature-control unit.

Temperature control is provided for the overall system, for operation of the overall system in extreme temperatures. In particular, the reagent reservoirs 91-97, the waste vessel 98 and the collecting container 36 have temperature control. This can be achieved, for example, by means of a second temperature-control unit 119, which is indicated by way of example as a heating coil in FIG. 8.

### IV.L. Detection Unit:

The detection unit 84 is provided at the end of the process chain. Depending on the nature of the sample processing, all known analysis methods can be integrated in the overall system.

The most important methods for detection and for analysis of biological molecules are mentioned in the following text:
PCR (polymerase chain reaction),
ELISA (enzyme-linked immunosorbent assay),
hybridization methods.

Section VI contains a more detailed description of the methods.

### IV.M. Control Unit:

The control unit 86 is used for controlling and monitoring the overall system. By way of example, a computer or a data processing appliance is provided as the control unit 86, in which the individual control steps for carrying out the detection method completely automatically are stored, in the form of control commands as software.

At the same time, data can be transferred via the control unit 86, for example via the Internet (online). The data transfer is used to match the results via a database, or for alarm production. The overall system can be controlled on line, thus allowing the system to be operated over relatively long distances.

### V. Extraction and Processing:

In the proposed overall system—apparatus 70—the enriched biological particles 13 are intended to be processed and/or decomposed, depending on the detection method. Extraction would have to be carried out for all molecular-biological analysis methods, in order to make the nucleic acids freely accessible. One sample is used (on an application-specific basis) for measurement of various parameters. A plurality of different extraction methods can be carried out simultaneously, successively or individually in the overall system. The following extraction methods can be integrated in the overall system:
chemical decomposition methods,
mechanical decomposition methods and/or
biochemical decomposition methods.

### V.A. Chemical Decomposition Methods:

Chemical decomposition can be carried out by means of chaotropic salts, in particular guanidinium hydrochloride or guanidinium thiocyanate. These are automatically received in the metering unit 41 (preferably syringe 42) by means of the described system, in order to decompose the particles 13 which are adhering to the beads 16.

### V.B. Mechanical Decomposition Methods:

Both decomposition via ultrasound and via heat are highly effective. For this purpose, the ultrasound appliance 56, 110 and/or a heating module—temperature-control unit 82 and/or 119—can be integrated in the overall system. Gravity forces (glass beads or tissue homogenizer) can also be used for extraction in the overall system. For this particular type of decomposition, the loaded beads 16 are transferred to a specific homogenizer, and effective decomposition can be carried out by friction forces between the glass beads and the homogenizer wall.

### V.C. Biochemical Decomposition Methods

One very successful method for decomposition of biological particles 13 is biochemical extraction. Enzymes, in particular proteases and RNases, can be used for biochemical extraction. Proteinase K is very frequently used for cell decomposition, lysozyme for bacteria decomposition.

### VI. Detection:

A multiplicity of detection methods have been established for detection of biological particles 13. One or more of the methods proposed here can be integrated in the proposed overall system—apparatus 70—depending on the requirement. One critical factor for analyte detection is measurement of defined panels corresponding to the fields of application. In addition, the overall system can be converted for methods which are still completely unknown.

The detection methods which are the most probable for integration in the overall system will be described in the following text:
PCR or real-time PCR,
ELISA or other immunological methods, and/or
hybridization methods.

### VI.A. PCR or Real-Time PCR:

The PCR method (polymerase chain reaction) is a method by means of which very small amounts of a DNA section can be amplified in a chain reaction. Nowadays, the PCR method is very often used when detections are intended to be made on the basis of specific DNA sequences, for example:
in forensics or for paternity tests,
in microbiology for detection of micro-organisms (bacteria and viruses),
in medical diagnostics, when the aim is to detect viral DNA or RNA in blood, or
in evolution biology, in order to track relationships and lines of descent.

In order to allow a PCR detection to be carried out, two short DNA pieces (primers) must be present, which match the sought DNA strand. The chain reaction which is started from them passes through up to 40 cycles, in which the amount of DNA is in each case doubled. The reaction can be observed directly and recorded quantitatively by the use of specific fluorescent samples (=oligonucleotides). This special form of PCR is referred to as real-time PCR, and is used for very rapid detection.

A further special form of PCR is reverse-transcription PCR (RT-PCT). This method is very frequently used for detection of viruses. Since most viruses have RNA instead of DNA as genetic material, it is absolutely essential for the RNA to be translated into DNA (reverse transcription). The actual detection can be carried out after the reverse transcription process, using normal PCR or real-time PCR.

### VI.B ELISA

ELISA (enzyme-linked immunosorbent assay) is a widely used method which allows the detection of specific proteins or other macromolecules (antigens). This is done using the mechanisms of the immune system. If the immune system identifies a substance as being foreign, it forms antibodies which dock with the foreign molecule, and thus label it. This so-called antibody-antigen interaction is used for the ELISA test. If the aim is to detect a specific protein, the antibodies which match it must be known, and must have been produced in advance by various recombinant methods or methods of all biology. If the sought protein is then present in a sample, it binds to the antibodies which have been immobilized on a carrier medium. After the antigen-antibody interaction, an enzyme-controlled reaction is initiated, which leads to a visible signal (color reaction, fluorescence or chemoluminescence). ELISA assays are nowadays widely used in medical diagnostics. However, they are also used in many other fields when the aim is to detect specific proteins or biological toxins. In the case of bacteria or virus detection, the specific surface protein is identified by the antibody.

### VI.C Hybridization Method

A DNA double helix can be imagined as a “zip fastener” (FIG. 9). The “teeth” of this zip fastener are the basis adenine (A), cytosine (C), guanine (G) and thymine (T). The information which the DNA contains is encrypted in the sequence of these four letters along the “zip fastener”.

In this case, opposite “teeth” only ever form AT or GC pairs. The sequence ACGCT, for example, is complementary to the base sequence TGCGA. Heating opens the “zip fastener”, thus resulting in individual strands. Short DNA pieces, which likewise are in the form of single strands, so-called probes, can now find their matching complementary piece on the long single strand. When these probes are cooled down, they bind to the appropriate site, and this is then referred to as hybridization. This can be made visible by labels (for example by means of a fluorescent dye). This makes it possible to find out whether specific sequences which, for example, represent specific genes, are or are not present in the DNA being examined. Widely known hybridization methods are in-situ hybridization, in particular fluorescence in-situ hybridization (FISH) and hybridization on microarrays.

### VII. Example of a Flowchart of the Overall System for Immune Detection (ELISA):

A flowchart for carrying out a detection method for detecting of particles in a fluid—in particular air—will be explained in more detail in the following text using the example of the ELISA method. A person skilled in the art can easily use this flowchart and its sub-sequences to appropriately configure the control unit 86, for example by programming.

The illustrated flowchart for the overall system includes a number of subsections (A-E, see below) which are required for immunodetection (ELISA). These subsections include basic commands with the aid of which the overall system can assume the appropriate positions.

All the basic commands and all the positions are first of all listed in the following text:

### Basic Commands

Syringe ↑; ↓
Piston ↑; ↓
go to Pos →; ← (go to)
Magnet on ←; Magnet off →
Linear-movement/pivoting unit [symbol] ↑; Linear-movement/pivoting unit ↓ [symbol] .
Air sampler on/off (Air sampler 30 on/off)
Ultrasound bath on/off
go to Position X
[detection unit on/off]
[temperature on/off]

### Positions (pos.):

The positions shown in the following text can be moved to by the linear drive 100:
Linear movement-pivoting unit position
Rest position
Magnetic beads position
Equilibration position
Decomposition 1 position
Decomposition 2 position
H₂O position
Cleaning position
Preservation solution
Waste position
Ultrasound bath position
Detection unit position

### VII.A. Equilibration of the System:

The following steps are carried out for equilibration. The commands which are automatically output by the control unit 86 are shown below.

1) Apparatus is in the rest position; syringe is filled with conversation solution.

Syringe in the rest position.

2) Emptying of the preservation solution into the waste.

Syringe ↑; go to Waste position→; Syringe ↓; Piston↓↑↓.

3) Rinsing with H₂O, 3×5 ml:

Syringe ↑; go to H2O position←; Syringe ↓; Piston ↑.

Syringe ↑; go to Waste position→; Syringe ↓; Piston ↓.

Syringe ↑; go to H2O position←; Syringe ↓; Piston ↓.

Syringe ↑; go to Waste position→; Syringe ↓; Piston ↓.

Syringe ↑; go to H2O position←; Syringe ↓; Piston ↑.

Syringe ↑; go to Waste position→; Syringe ↓; Piston ↓.

4) Rinsing with equilibration solution, 3×5 ml:

Syringe ↑; go to Equilibration solution position←; Syringe ↓; Piston ↑.

Syringe ↑; go to Waste position→; Syringe ↓; Piston ↓.

Syringe ↑; go to Equilibration solution position←; Syringe ↓; Piston ↑.

Syringe ↑; go to Waste position→; Syringe ↓; Piston ↓.

Syringe ↑; go to Equilibration solution position←; Syringe ↓; Piston ↑.

Syringe ↑; go to Waste position→; Syringe ↓; Piston ↓.

5) Syringe returns to the Rest position:

Syringe ↑; go to Rest position←.

VII.B. Loading of the Air Sampler with Magnetic Beads 1×5 ml.

The following commands are carried out, controlled by the control device, in order to load the air sampler 30:

Syringe ↑; go to Position mag. Beads←; Syringe ↓; Piston ↑↓↑.

Linear-movement/pivoting unit ↓

[symbol]

Syringe ↑; go to Linear-movement/pivoting unit position←; Syringe ↓; Piston ↓.

Syringe ↑; go to Rest position←; Syringe ↓.

### VII.C. “Sampling” and Detection Using ELISA

The following steps are carried out using the respectively indicated command sequences, for sampling and for detection:

1) Start sampling

Linear-movement/pivoting unit

[symbol]

Air sampler on.

2) End sampling

Air sampler off.

Linear-movement/pivoting unit

[symbol]

3) Magnet to the syringe:

Magnet on←

4) Draw 1×5 ml magnetic beads with syringe from the air sampler

Syringe ↑; go to Linear-movement/pivoting unit position←; Syringe ↓; Piston ↓↑↓↑.

5) 4 ml (from 1×5 ml) to the waste.

Syringe ↑; go to Waste position→; Syringe ↓; Piston ↓

6) Magnet away from the syringe.

Magnet off→.

7) Receipt of 4 ml equilibration solution

Syringe ↑; go to Equilibration solution position←; Syringe ↓; Piston ↑.

8) Magnet to the syringe.

Magnet on←.

9) Syringe 3 ml of equilibration solution into the air sampler

Tip ↑; go to Linear-movement/pivoting unit position←; tip ↓; Piston ↓.

10) Reception of the 3 ml of equilibration solution into the syringe again

Syringe ↑; Syringe ↑; Piston ↓↑↓↑.

11) first repetition of steps 5-10.

12) second repetition of steps 5-10.

13) reduction of the volume to 10 μl.

Syringe ↑; go to Waste position→; Syringe ↓; Piston ↓,

14) Magnet away from the syringe.

Magnet off→.

15) 10 μl beads into the detection unit; Syringe ↓; Piston ↓↑↓↑↓.

16) rinsing of the syringe with H₂O.

Routine VII.A.3): Rinsing with H₂O, 3×5 ml.

Syringe ↑; go to Rest position←; Syringe ↓.

17) start of detection (ELISA) in the detection unit.

Detection unit on.

### VII.D. Cleaning

The following steps are carried out, by means of the stated control commands, for cleaning:

1) rinsing of the syringe with H₂O, 3×5 ml.

Routine VII.A.3): rinsing with H₂O, 3×5 ml.

2) cleaning with cleaning agent, 3×5 ml.

Syringe ↑ go to Cleaning position→; Syringe ↓; Piston ↑.

Syringe ↑ go to Waste position→; Syringe ↓; Piston ↓.

Syringe ↑ go to Cleaning position→; Syringe ↓; Piston ↑.

Syringe ↑ go to Waste position→; Syringe ↓; Piston ↓.

Syringe ↑ go to Cleaning position→; Syringe ↓; Piston ↑.

Syringe ↑ go to Waste position→; Syringe ↓; Piston ↓.

3) rinsing of the syringe with H₂O, 6×5 ml.

Routine VII.A.3): rinsing with H₂O, 3×5 ml

Routine VII.A.3): rinsing with H₂O, 3×5 ml

Syringe ↑; go to Rest position←; Syringe ↓.

E) Preservation

1) rinsing with H₂O, 3×5 ml.

2) rinsing with preservation solution 3×5 ml.

Syringe ↑ go to Preservation position→; Syringe ↓; Piston ↑.

Syringe ↑ go to Waste position→; Syringe ↓; Piston ↓.

Syringe ↑ go to Preservation position→; Syringe ↓; Piston ↑.

Syringe ↑ go to Waste position→; Syringe ↓; Piston ↓.

Syringe ↑ go to Preservation position→; Syringe ↓; Piston ↑.

Syringe ↑ go to Waste position→; Syringe ↓; Piston ↓.

Syringe ↑ go to Rest position←; Syringe ↓.

Respective sampling process is completed by transferring the beads and the lysate into or to an appropriate detection unit 84. This can be done in particular by injection onto a microfluidic disk.

A further option is to apply the beads 16 to a membrane, preferably to a micromechanical filter (not illustrated) whose surface can then be used as a detection platform. Detection on the surface of a micromechanical filter has already been described in detail in German patent applications 10 2006 026 559.5 and in 10 2007 021 387.7. Furthermore, a refined method is the subject matter of a German patent application, submitted on the same date as this application, entitled “Optischer Partikelfilter sowie Detektionsverfahren” [Optical particle filters and detection methods], for which EADS Deutschland GmbH is likewise the applicant. For further details, reference is made expressly to the abovementioned further patent applications.

### VIII. Further Alternatives and Applications

VIII.A Enrichment of Particles from Liquids

Enrichment of biological particles from liquids is also possible by minor modifications to the overall system proposed here. For this purpose, as is illustrated in FIG. 10, the air sampler 30 is replaced by a filtration unit 120, which allows a high liquid flow rate.

The figure shows the design of a flow cell 122 which is bounded by two membranes 124, 126. The pore size of the membranes 124, 126 should be chosen such that the particles 13, in particular micro-organisms, can pass through, but the paramagnetic particles 16 are retained. The paramagnetic beads 16, to which the particles 13 bind effectively, are located between the membranes 124, 126.

In order to ensure a homogeneous distribution of the paramagnetic beads 16, a stirrer 128 (rotor), which is driven by the through-flow 134, is located in the flow cell 122.

The beads 16 are removed automatically through a closure in the flow cell 122, in particular through a septum 130 which can be pierced by an cannula 132.

### VIII.B. Use of Alternative Beads

It is also feasible to use non-paramagnetic beads in the overall system. The beads could be enriched after the “air sampling” step instead of by means of a magnetic field via a porous membrane, preferably a micromechanical filter. This filter would retain the beads, but would allow liquids to pass through. In consequence, all the washing and detection solutions which are required for immunodetection (ELISA) are pumped through these micromechanical filters.

Examples of micromechanical filters and detection methods which can be carried out using them, as well as detection apparatuses which have such filters can be found in German patent application 10 2006 026 559.9, German patent application 10 2007 021 387.7, and the two German patent applications submitted in parallel with the present patent application, for which the (joint) applicant is EADS Deutschland GmbH, entitled “Optischer Partikelfilter sowie Detektionsverfahren” and “Partikelfilter sowie Herstellverfahren hierfür” [Optical particle filters and detection methods] [Particle filters and production methods for them]. Reference is made to these patent applications for further details.

VIII.C. Use of Beads with Nucleic Acids or of a Silica Matrix

A further option for sampling of micro-organisms is to use nucleic-acid-coupled beads in the overall system. For this purpose, the micro-organisms from the air are enriched and are decomposed (for extraction methods, see above). After the extraction, the beads which have been coated with nucleic acids are passed to the lysate, and the genetic material of the micro-organisms can be hybridized with the nucleic acid on the beads. Non-specific binding of the extracted DNA to a silica matrix is also possible. For this purpose, after cell lysis, an appropriate amount of silica matrix is added to the lysate, and the DNA released can bind non-specifically to the silica particles.

VIII.D. Separation of the Antibody-Antigen Complex from the Beads

With certain detection methods (for example ELISA), it is necessary to separate the antibody-antigen complexes as shown in FIG. 1 from the beads. The following cleavage options can be carried out with the overall system:
chemical cleavage by destruction of the biotin-streptavidin bond
chemical cleavage by use of sulfo-NHS-SS biotin
thermal cleavage by denaturing in the area of the boiling point
biochemical cleavage by use of suitable proteases (for example papain)
physical exposure to light. One precondition for this is the use of a light-sensitive biotin linker which breaks down when exposed to light at a specific wavelength.

Subsequent detection could then be carried out using traditional molecular-biological methods (PCR or hybridization, see above).

### VIII.E. Integration of Other Air Samplers

The air sampler from the SKC Company was integrated in the overall system proposed here. The flexible and modular configuration of the overall system also allows the integration of other types of air sampler, however (see the publication Hogan et al. 2005). The following air sampler types would likewise be suitable for integration in the overall system:
AGI-30 (all-glass impinger)
Frit bubbler or
Coriolis Air Sampler (Bertin Company).

### VIII.F Automatic Cleaning/Disinfection

A fully automatic cleaning and disinfection program can be established in the overall system. All feasible cleaning and disinfection solutions can be used in the robust system. The following solutions are preferably used:
Acids,
Lyes,
Detergents and/or
Alcohols.

For this purpose, all components of the air sampler 30 are automatically cleaned by supplying the solutions. A rinsing step is then possible. This is carried out by means of a fluidic system which provides the reagents, applies them or introduces them, and then reabsorbs them.

After the cleaning of the air sampler and of the entire fluidic system by the liquids mentioned above, the system is disinfected by UV radiation. For this purpose, a plurality of UV tubes are placed above the system, and sterilize the overall system in a relatively short time period.

### VIII.G Applications of the Overall System

The overall system opens up a wide range of applications. The following application options are feasible:
medical applications for diagnostics, in particular rapid detection of pathogens of infectious diseases from bodily fluids, in particular blood, saliva, lacrimal fluid and urine;
military applications, in particular integration of the overall system in military vehicles, marine vessels, submarines and airborne vehicles;
use as a mobile system in all military and civil fields;
use in the field of “Homeland Security”, in particular for defence against terrorist attacks using biological weapons;
detection of explosives, particularly for defense against terrorist attacks;
detection of various narcotics and drugs, in particular for use with the police, federal police and railway police;
civil applications, in particular for monitoring of foodstuffs, monitoring of drinking water and in building biology (room-air monitoring);
aerospace applications, in particular for checking the on-board water and the cabin air, and in conjunction with space missions, in particular for finding traces of extra terrestrial life.

### LIST OF REFERENCE SYMBOLS

10 Antibody

11 Centre axis

12 Antigens

13 Particles (in particular micro-organism)

14 Surface

15 Tangent

16 Bead

18 Biotin

20 Streptadivin

22 F_{C}-Domain

24 Phage protein

26 Bacterium

30 Air sampler

32 Inlet

34 Outlet

36 Collecting container

38 Tangential nozzles

39 Air flow

40 Collecting liquid (enrichment liquid)

41 Metering unit

42 Syringe

43 Metering volume

44 Magnet

45 Outlet channel

46 Outer wall

48 Inner wall

50 (hollow) syringe piston

52 Magnet piston

54 Piston base

56 Ultrasonic appliance

58 Bar magnet

60 Membrane

62 Further vessel

64 Nozzle attachment

66 Linear-movement/pivoting unit

70 Apparatus (overall system)

72 Collecting device

74 Transfer unit

76 Group of reservoirs

78 Drive unit

80 Decomposition device

82 Temperature-control unit

84 Detection unit

86 Control unit

91 First reservoir (bead; solution with paramagnetic beads)

92 Second reservoir (equilibration solution)

93 Third reservoir (first decomposition solution)

94 Fourth reservoir (second decomposition solution)

95 Fifth reservoir (collecting liquid, for example water, H₂O)

96 Sixth reservoir (cleaning solution)

97 Seventh reservoir (preservation solution)

98 Waste vessel

99 Rest position

100 Linear drive

102 Unit for holding the metering unit

104 First motor

106 Second motor

108 Third motor

110 Ultrasound bath

112, Z1 First movement (syringe in the Z direction)

114, Z2 Second movement (syringe piston in the Z direction)

116, Z3 Third movement (magnet in the Z direction)

118 Return flow to the pump

120 Filtration unit

122 Flow cell

124 Membrane

126 Membrane

128 Stirrer

130 Septum (closure)

132 Cannula

134 Through-flow

140 Microliter pipette

142 Pipette tip

144 First suction unit

145 Second suction unit

146 Pipetting system

1. An apparatus for automatic detection of particles comprising:
a collecting device onfigured to contain a collecting fluid including separating particles that are selectively bound to the particles provided from a particle-fluid mixture;
a separating device configured to separate from the collecting fluid those of the separating particles that are bound to particles; and
a detecting device configured to detect of an amount of the particles based on the separating particles that are separated from the collecting fluid by the separating device.

2. The apparatus as claimed in claim 1, further comprising
a metering unit configured to provide the collecting fluid to the collecting device.

3. The apparatus as claimed in claim 2, wherein
the metering unit is configured to selectively retain the separating particles in the metering unit or provide the separating particles in the collecting fluid to the collecting device.

4. The apparatus as claimed in claim 2, wherein
the metering unit includes at least one of a syringe and a pipette.

5. The apparatus as claimed in claim 2, wherein
the metering unit is configured for movement by a drive unit between the collecting device and the detecting device.

6. The apparatus as claimed in claim 1, further comprising
a plurality of reservoirs configured to provide different fluids for use by the detecting device during detection of the particles.

7. The apparatus as claimed in claim 6,
wherein the reservoirs are configured to contain at least one of the following:
separating particle body solution,
equilibration solution,
decomposition solution,
the collecting fluid,
cleaning solution, and
preservation solution.

8. The apparatus as claimed in claim 6, further comprising
a waste vessel associated with the plurality of reservoirs.

9. The apparatus as claimed in claim 5, wherein
the drive unit is configured to selectively move the metering unit to the reservoirs.

10. The apparatus as claimed in claim 6, wherein
the reservoirs are temperature-controlled.

11. The apparatus as claimed in claim 5, wherein
the collecting can device is configured to be filled automatically with the collecting fluid.

12. The apparatus as claimed in claim 11, wherein
the metering unit is configured for movement by the drive unit to automatically fill the collecting device.

13. The apparatus as claimed in claim 1, further comprising
at least one controllable motor configured to drive a metering unit for metered reception or delivery of liquids to or from the collecting device.

14. The apparatus as claimed in claim 1, wherein
the separating device comprises a magnet that is configured to magnetically interact with the separating particles.

15. The apparatus as claimed in claim 14, wherein
the magnet disposed on at least one of a wall of a metering chamber of a metering unit and on a piston base of a piston of the metering unit, the metering unit being configured to provide the collecting fluid to the collecting device.

16. The apparatus as claimed in claim 14, wherein
the magnet includes a permanent magnet that is selectively moveable to a first position for retaining the separating particles and to a second position for releasing the separating particles.

17. The apparatus as claimed in claim 1, wherein
the separating device includes a micromechanical filter defining pores having diameters that are greater than a diameter of the particles and less than a diameter of the separating particles.

18. The apparatus as claimed in claim 1, wherein
the collecting device includes a gas collecting device including an air sampler that is configured to transfer the particles from a gas which contains the particles into the collecting fluid that contains the separating particles.

19. The apparatus as claimed in claim 18, wherein
the gas collecting device includes at least one nozzle that communicates with the collecting fluid and is configured to introduce the gas into the collecting fluid.

20. The apparatus as claimed in claim 1, wherein
the collecting device includes a collecting container that is configured to contain the collecting fluid and further configured for movement by means a transfer unit between a collecting position in which a particle-fluid mixture is passed through the collecting fluid and a position collecting container contains the collecting fluid.

21. The apparatus as claimed in claim 20,
the transfer unit includes a moving unit that is configured to move the collecting container.

22. The apparatus as claimed in claim 1, wherein
the collecting device includes a filtering unit that includes a flow cell which is partitioned by membranes and contains the separating particles, the membranes being configured to enable particle-liquid mixture to pass therethrough, such that the membranes are permeable for the particles and are impermeable for the particles.

23. A method for detecting particles in a particle-fluid mixture comprising:
collecting the particles in a collecting fluid that contains separating particles which bind to specific ones of the particles, such that the separating particles bind to the particles while the particles are collected;
separating the separating particles to which the particles are bound from the collecting fluid; and
detecting an amount of the particles based on the separating particles separated during the separating operation.

24. The method as claimed in claim 23, wherein
the separating includes filling a metering unit in a metered manner with the collecting fluid that includes the separating particles and the particles which are bound to the separating particles and with a liquid used for extraction and enrichment of the separating particles.

25. The method as claimed in claim 24, wherein
the separating further includes retaining the separating particles in the metering unit, while the liquid is delivered from the metering unit.
